(19) United States
(12) Patent Application Publication
(10) Pub. No.: US 2007/0116713 A1
(43) Pub. Date: May 24, 2007

(54) Compositions and methods for reducing or controlling blood cholesterol, lipoproteins, triglycerides and atherosclerosis

(76) Inventors: Robert P. Gill, St. Mary's, OH (US); Lorraine M. Kaup, Fort Recovery, OH (US)

Correspondence Address: WALKER & JOCKE, L.P.A., 231 SOUTH BROADWAY STREET, MEDINA, OH, 44256, US

(21) Appl. No.: 11/285,894

(22) Filed: Nov. 22, 2005

(51) Int. Cl. A61K 36/06; A61K 31/555; A61K 31/525; A61K 31/205

(52) U.S. Cl. 424/195.16; 424/702; 514/184; 514/251; 514/554; 514/356

(57) ABSTRACT

Compositions and methods related to the administration of red yeast rice, nicotinic acid, L-carnitine, folic acid, chromium picolinate, and selenium to reduce or control blood cholesterol, triglycerides, and lipoproteins in a mammal, and to reduce inflammation associated with coronary artery disease.

### FIELD OF INVENTION

The invention relates to compounds and methods that reduce or control levels of cholesterol and triglycerides, thus preferably inhibiting or arresting the development of atherosclerosis when administered to mammals, including humans.

### BACKGROUND OF THE INVENTION

Approximately seven million men and women in America have coronary artery disease. Of these, about 1.25 million will have a heart attack which will be fatal for 500,000 of these individuals. It is believed the second most significant risk factor for coronary heart disease after hypertension is hypercholesteremia. Several large trials have shown cholesterol reduction therapy to be effective for “primary prevention” of coronary artery disease. However, this concept is flawed because most people who start taking therapeutic drugs already have atherosclerosis. The drug treatment is thus secondary prevention. There is evidence that the disease is multi-factorial and current drug treatments may play many different roles in alleviating the disease process.

A popular treatment for coronary artery disease includes the use of statin drugs. There is evidence that the statin drugs work in various ways. They can be of benefit through various atherosclerotic and antithrombotic mechanisms that may be independent of lowering LDL-C. Some of these effects are improved endothelial function, plaque stabilization, decreased lipoprotein oxidation, improved platelet function, reduced blood viscosity, and suppression of inflammations. See R. S. Rosenson & C. C. Tangney, Antiartherothrombotic Properties of Statins: Implications for Cardiovascular Event Reduction, 279 JAMA 1643 (1998) and R. S. Blumenthol, Statins: Effective Antiartherosclerotic Therapy, 139 AMERICAN HEART JOURNAL 577 (2000). As prior experience has shown, drug therapy is an ever-changing process that differs genetically and environmentally for each individual.

Although there is a strong relationship between age and the development of coronary artery disease, it has been well documented that atherosclerosis happens at a very early age. See S. M. Grundy, T. Bazzarre, & J. Cleeman et al., Beyond Secondary Prevention: Identifying the High Risk Patient for Primary Prevention, 101 CIRCULATION 111 (2000). Autopsy reports of young servicemen, and from donated hearts in young adults confirm this result. See J. J. McNamara, M. A. Molot, & J. F. Stremple et al., Coronary Artery Disease in Combat Casualties in Vietnam, 216 JAMA 1185 (1971); W. F. Enos, Jr., J. C. Beyer, & R. H Holmes, Pathogenesis of Coronary Artery Disease in American Soldiers killed in Korea, 158 JAMA 912 (1955); E. Tutor, S. R. Kapadra, & K. M. Ziada, et al., Coronary Atherosclerosis Begins at Young Age: Intravascular Ultrasound Evidence of Disease in Individuals <20 Years Old, 100 CIRCULATION 1524 (Supple. 18) (1999). There are cases of young adults, less than 50, with significant coronary artery disease and myocardial infarction, even with normal LDLs. See K. O Akosah, R. Cerniglia, & P. Havlik et al., Myocardial Infarction in Young Adults with LDL Cholesterols <100 mg/dl: Clinical Profile and 1-year Outcomes, CHEST IN PRESS.

Further, inflammation appears to be an important factor in heart disease and stroke. One of the markers used recently to detect inflammation is called high sensitivity CRP (C-reactive protein). Strong evidence indicates that CRP predicts future cardiovascular events. See P. M. Ridker, Clinical Application of C-reactive Protein for Cardiovascular Disease Detection and Prevention, 107 CIRCULATION 363-69 (2003); P. M. Ridker, High Sensitivity C-reactive Protein Potential Adjunct for Global Risk Assessment in the Primary Prevention of Cardiovascular Disease, 103 CIRCULATION 1813-18 (2001); P. M. Ridker, C. H. Hennekens, J. E. Buring, & N. Rifai., C-reactive Protein and Other Markers of Inflammation in the Prediction of Cardiovascular Disease in Women, 342 N. ENG. J. MED. 836-43 (2000).

In the United States millions of individuals are being treated with statin drugs for high cholesterol. The statin drugs may be a part of the solution to the problem of coronary artery disease and myocardial infarction, but the complex nature of the problem suggests that other processes need to be addressed as well.

Additionally, statin drugs are not the answer for everyone. Statin drugs come with a high price tag and undesirable side effects. For some patients, the drugs may be ineffective. For some patients, statin drugs affect only a narrow portion of their medical needs. And, some patients are adverse to taking high-powered drugs.

Thus there exists a need in the art for a therapy that addresses the problems presented above while providing better and more advantageous results.

### SUMMARY OF THE INVENTION

Exemplary embodiments of the invention present an improved strategy to decrease cholesterol, increase HDL, decrease triglycerides, and reduce the inflammatory process associated with coronary artery disease.

It is an object of exemplary embodiments to provide a cholesterol treatment composition comprising an effective amount of red yeast rice, nicotinic acid, L-carnitine, folic acid, chromium picolinate, and selenium. In an exemplary composition, the red yeast rice may be produced from the fermentation of at least one member selected from the group consisting of Monascus purpureus, Monascus rubber, Monascus fuliginosus, Monascus Pilosus, and Monascus albidus.

The exemplary composition comprises, in relative amounts, about 300 parts red yeast rice, about 20 to about 62.5 parts nicotinic acid, about 100 parts L-carnitine, about 0.15 parts folic acid, about 0.05 parts chromium picolinate, and about 0.0125 parts selenium. The exemplary composition may include an inactive pharmaceutically acceptable carrier.

In the exemplary composition, the parts are milligrams, and the composition is in a dosage form of tablets or capsules. The dosage form may be a capsule and the effective amount may comprise one (1) to four (4) capsules per day.

Exemplary embodiments of the composition may include low-dose nicotinic acid or high-dose nicotinic acid. An exemplary low-dose nicotinic acid composition comprises, in relative amounts: about 300 parts red yeast rice, about 20 parts nicotinic acid, about 100 parts L-carnitine, about 0.15 parts folic acid, about 0.05 parts chromium picolinate, and about 0.0125 parts selenium. An exemplary high-dose nicotinic acid composition comprises, in relative amounts: about 300 parts red yeast rice, about 62.5 parts nicotinic acid, about 100 parts L-carnitine, about 0.15 parts folic acid, about 0.05 parts chromium picolinate, and about 0.0125 parts selenium.

An exemplary embodiment of the invention provides a method of lowering cholesterol. The exemplary method includes evaluating a cholesterol level of a patient, and responsive to the cholesterol level, administering a composition comprising about 300 parts red yeast rice; about 20 parts to about 62.5 parts nicotinic acid; about 100 parts L-carnitine; about 0.15 parts folic acid; about 0.05 parts chromium picolinate; and about 0.0125 parts selenium, in therapeutically effective amounts to the patient in need of such treatment.

In the exemplary method, the therapeutically effective amount may be a daily dosage of between about 300 mg to about 1200 mg red yeast rice, about 20 mg to about 250 mg nicotinic acid, about 100 mg to about 400 mg L-carnitine, about 0.15 mg to about 0.6 mg folic acid, about 0.05 mg to about 0.2 mg chromium picolinate, and about 0.0125 mg to about 0.05 mg selenium. The therapeutically effective amount may be administered daily by ingestion of one (1) to four (4) capsules.

### DESCRIPTION OF EXEMPLARY EMBODIMENTS

An exemplary form of the invention provides a combination of ingredients believed to present an innovative strategy to decrease cholesterol, increase HDL, decrease triglycerides, and/or reduce the inflammation associated with coronary artery disease and myocardial infarction. It is believed that the ingredients provide synergistic effects in the patient.

One ingredient in an exemplary composition comprises monacolin K derived from Red Yeast Rice. The Red Yeast is derived from Monascus purpureos west and/or other monascus species. The red rice product is the result of fermentation using a mixture of Monascus fungi. Monacolin K and other monacolin-like substances inhibit the function of HMG-CoA reductase, which is an enzyme important for the production of cholesterol in the body.

Another ingredient in the exemplary composition is niacin, also called vitamin B₃, or nicotinic acid. Niacin is one of eight water-soluble B vitamins. These B vitamins help the body to convert carbohydrates into glucose, which is used to produce energy. These vitamins also are essential in the breakdown of fats and proteins. Niacin is known to play an important role in ridding the body of toxic and harmful chemicals. It is also known to be effective in improving circulation and reducing cholesterol levels in the blood, although the mechanisms are not fully understood.

Another ingredient in the exemplary composition is L-Carnitine, a derivative of the amino acid, lysine. L-Carnitine is a naturally occurring amino acid derivative found in meat and dairy products. L-Carnitine may be synthesized in the body from the amino acids lysine and methionine. Deficiency in L-Carnitine can produce muscle fatigue, cramps, changes in kidney function following exercise, premature aging, and heart beat irregularities in someone who has had a heart attack.

One of the major side effects of the statin drugs is the associated myopathy. Statins, such as Lovastatin and other inhibitors of HMG-CoA reductase can cause myopathies, which are manifested by muscle pain or weakness. Mild muscle pain may be exhibited without significant rise in creatinine kinase, while significant pain can be accompanied by a significant rise in creatinine kinase. U.S. Pat. No. 6,245,800 discloses the simultaneous administration of L-Carnitine with levels of Lovastatin that normally cause side effects in order to decrease levels of creatinine kinase.

Another ingredient in the exemplary composition is chromium. Chromium, and in particular, chromium picolinate, has been shown to facilitate reductions in triglycerides (TC) and LDL serum cholesterol, especially in those people whose baseline TC levels were above 200 mg/dl. See Gilbert R. Kaats, PhD, Samuel C. Keith, John A. Wise, PhD, Dennis Pullin, MS, & William G. Squires, Jr. PhD., Effects of Baseline Total Cholesterol Levels on Diet and Exercise Interventions, 2(10) J. AM. NUTRACEUTICAL ASSOC. 42-49 (1999). Chromium picolinate can beneficially lower LDL and its transport protein, apolipoprotein B, while increasing HDL and apolipoprotein. See A. Raymond I. Press, MD, Jack Geller, MD, & Gary Evans, PhD., The Effect of Chromium Picolinate on Serum Cholesterol and Apolipoprotein Fractions in Human Subjects, 152 W.J. OF MED. 41-45 (1990).

Chromium has the additional benefit of addressing impaired glucose tolerance and insulin resistance in diabetic patients. Diabetes mellitus along with hyperlipidemia and hypertension increases the risk of heart attack and stroke. Thus, this ingredient is believed to provide synergistic effects.

Another ingredient in the exemplary composition is folic acid. This nutrient may provide benefits by decreasing homocysteine, an amino acid in the blood that attacks blood vessel walls and promotes cardiovascular disease. It has been reported that the incidence of restenosis in smaller coronary arteries could be markedly reduced by decreasing homocysteine levels through supplementation with folic acid and vitamins B₆ and B₁₂. See Guido Schnyder et al., Effect of Homocysteine-lowering Therapy on Restenosis after Percutaneous Coronary Intervention for Narrowing in Small Coronary Arteries, 91 AM. J. OF CARDIOLOGY, 1265-69 (2003). It has also been surmised that folic acid deficiencies are associated with substantially increased risk of dying from cardiovascular disease. See Catherine M. Loria, et al., Serum Folate and Cardiovascular Disease Mortality among US Men and Women, 160 ARCHIVES OF INTERNAL MEDICINE 2358-62 (2000). Furthermore, folic acid is essential for the synthesis of adenine and thymine, two of the four nucleic acids that make up human genes, DNA, and chromosomes. Folic acid deficiency has been clearly linked to elevated levels of homocysteine which in turn has been linked to cardiovascular disease.

Another ingredient in the exemplary composition is selenium, a trace mineral essential to good health. Selenium is incorporated into proteins to make selenoproteins. These agents act as antioxidant enzymes to help prevent cellular damage from free radicals. The free radicals are a by-product of oxygen metabolism. Free radicals may contribute to the development of chronic disease, including heart disease. See S. B. Goldhaber, Trace Element Risk Assessment: Essentiality v.s. Toxicity, 38 REGULATORY TOXICOLOGY AND PHARMACOLOGY 232-42 (2003); G. F. Combs, Jr. & W. P. Gray, Chemopreventive Agents: Selenium, 79 PARMACOL THER., 179-92 (1998). It is thought that selenium is one of the antioxidants that prevents the build up of plaques in the coronary arteries by limiting the oxidation of LDL cholesterol. See J. Neve, Selenium as a Risk Factor for Cardiovascular Disease, 3 J. CARDIOVASC. RISK 42-47 (1996).

### EXAMPLE 1

An exemplary composition includes:
about 30.00 g red yeast rice;
about 6.250 g nicotinic acid;
about 10.00 g L-carnitine;
about 0.015 g folic acid;
about 0.005 g chromium picolinate; and
about 0.00125 g selenium; and
about 4.5 g pharmaceutically acceptable filler, such as microcrystalline cellulose.

In an exemplary embodiment, the above-listed powders were titrated together and 100 capsules were filled. Each capsule contained:
about 300 mg red yeast rice;
about 62.5 mg nicotinic acid;
about 100 mg L-carnitine;
about 0.15 mg folic acid;
about 0.05 mg chromium picolinate; and
about 0.0125 mg selenium.

Exemplary dosages of the composition are from one (1) to four (4) capsules daily. Thus, exemplary daily intakes range from:
about 300 mg-1200 mg red yeast rice;
about 62.5 mg-250 mg nicotinic acid;
about 100 mg-400 mg L-carnitine;
about 0.15 mg-0.6 mg folic acid;
about 0.05 mg-0.2 mg chromium picolinate; and
about 0.0125 mg-0.05 mg selenium.
   Case Study:

An initial study included 34 subjects. The subjects were advised to exercise and follow a strict low-fat diet, in addition to consuming doses of the exemplary composition. The subjects were tracked from 8 to 24 months. Twenty-eight of the 34 participants had a favorable outcome with minimal, if any, side effects. There was a 12.9% decrease in total cholesterol and a 17.9% decrease in LDL among those subjects who benefitted from the program. Table 1 provides dosages and effects on total cholesterol, triglycerides, LDL, and HDL levels for each of the subjects.

In two of the subjects, the nicotinic acid was not tolerated, and was removed from the formulation as indicated in Table 1 below. It is contemplated within the scope of the invention to provide alternate formulations which eliminate nicotinic acid for use by those persons who cannot tolerate it.

TABLE 1

| Identifier | Dosage | Cholesterol | Triglycerides | LDL | HDL | % Δ Chol. | % Δ LDL |
|---|---|---|---|---|---|---|---|
| 1 | 3 QD | 239 | 103 | 157 | 61 | 23 | 28 |
| | | 228 | 150 | 139 | 59 | | |
| | | 183 | 61 | 113 | 58 | | |
| 2 | 3 QD | 236 | 157 | 150 | 54 | 18 | 17 |
| | | 204 | 134 | 120 | 57 | | |
| | | 194 | 87 | 125 | 51 | | |
| 3 | 2 QD | 259 | 101 | 194 | 45 | 20 | 29 |
| | | 207 | 56 | 138 | 58 | | |
| 4 | 3 QD | 253 | 154 | 160 | 62 | 9 | 3 |
| | | 231 | 55 | 156 | 64 | | |
| 5 | 3 QD | 242 | 124 | 167 | 50 | 17 | 43 |
| | | 193 | 118 | 121 | 48 | | |
| | | 201 | 271 | 95 | 51 | | |
| 6 | 2 QD | 194 | 70 | 140 | 40 | 1 | 5 |
| | | 172 | 101 | 111 | 40 | | |
| | | 193 | 118 | 133 | 36 | | |
| 7 | 1 *WO | 288 | 129 | 196 | 66 | 24 | 32 |
| | | 214 | 91 | 123 | 72 | | |
| | | 220 | 86 | 134 | 68 | | |
| 8 | 2 *WO | 269 | 256 | 178 | 39 | 12 | 28 |
| | | 238 | 256 | 149 | 37 | | |
| | | 236 | 322 | 129 | 42 | | |
| 9 | 2 QD | 207 | 205 | 128 | 38 | 3 | 3 |
| | | 203 | 131 | 134 | 43 | | |
| | | 200 | 164 | 124 | 43 | | |
| 10 | 3 QD | 238 | 328 | 125 | 48 | 15 | 6 |
| | | 216 | 88 | 148 | 50 | | |
| | | 202 | 157 | 117 | 53 | | |
| 11 | 2 QD | 172 | 141 | 85 | 58 | −26 | −48 |
| | | 216 | 177 | 130 | 50 | | |
| | | 216 | 153 | 126 | 59 | | |
| 12 | 2 QD | 235 | 239 | 117 | 70 | 3 | 6 |
| | | 223 | 231 | 83 | 93 | | |
| | | 229 | 245 | 110 | 70 | | |
| 13 | 2 QD | 305 | 218 | 173 | 88 | 12 | 2 |
| | | 212 | 103 | 110 | 81 | | |
| | | 268 | 113 | 169 | 76 | | |
| 14 | 3 QD | 231 | 149 | 156 | 45 | 14 | 24 |
| | | 204 | 122 | 126 | 53 | | |
| | | 198 | 168 | 118 | 46 | | |
| 15 | 3 QD | 210 | 51 | 138 | 61 | 18 | 25 |
| | | 208 | 79 | 129 | 63 | | |
| | | 173 | 70 | 104 | 55 | | |
| 16 | 3 QD | 227 | 145 | 123 | 65 | 10 | 15 |
| | | 206 | 125 | 112 | 69 | | |
| | | 204 | 95 | 105 | 80 | | |
| 17 | 1 QD | 232 | 59 | 136 | 84 | 29 | 29 |
| | | 195 | 90 | 99 | 78 | | |
| | | 210 | 72 | 126 | 69 | | |
| 18 | 2 QD | 249 | 93 | 163 | 67 | 9 | 7 |
| | | 212 | 70 | 133 | 65 | | |
| 19 | 2 QD | 231 | 329 | 118 | 47 | 15 | 18 |
| | | 213 | 207 | 127 | 45 | | |
| 20 | 2 QD | 235 | 107 | 124 | 90 | 8 | −8 |
| | | 177 | 109 | 72 | 83 | | |
| | | 207 | 86 | 98 | 92 | | |
| 21 | 2 QD | 233 | 62 | 160 | 61 | 12 | 21 |
| | | 181 | 57 | 101 | 69 | | |
| 22 | 2 QD | 227 | 463 | — | 42 | 22 | 37 |
| | | 221 | 272 | 110 | 56 | | |
| | | 208 | 295 | 100 | 49 | | |
| 23 | 2 QD | 213 | 122 | 139 | 49 | 8 | 10 |
| | | 236 | 97 | 169 | 47 | | |
| | | 244 | 120 | 177 | 43 | | |
| 24 | 1 QD | 231 | 87 | 139 | 74 | −15 | −27 |
| | | 224 | 126 | 137 | 61 | | |
| | | 262 | 78 | 172 | 74 | | |
| 25 | 4 QD | 240 | 165 | 153 | 54 | −13 | −24 |
| | | 199 | 104 | 134 | 44 | | |
| | | 220 | 176 | 145 | 39 | | |
| 26 | 4 QD | 230 | 118 | 166 | 40 | 8 | 5 |
| | | 225 | 143 | 151 | 45 | | |
| | | 191 | 191 | 108 | 44 | | |
| 27 | 2 QD | 275 | 217 | 154 | 78 | 17 | 35 |
| | | 231 | 183 | 133 | 61 | | |
| | | 259 | 146 | 162 | 67 | | |
| 28 | 2 QD | 269 | 216 | 177 | 48 | 6 | −5 |
| | | 212 | 104 | 139 | 52 | | |
| | | 226 | 207 | 126 | 58 | | |
| 29 | 2 QD | 254 | 368 | 130 | 50 | 16 | 29 |
| | | 266 | 307 | 153 | 51 | | |
| | | 291 | 565 | — | 51 | | |
| 30 | 1 QD | 228 | 140 | 141 | 59 | −15 | −18 |
| | | 223 | 139 | 135 | 60 | | |
| 31 | 4 QD | 237 | 86 | 154 | 65 | 2 | 5 |
| | | 233 | 72 | 148 | 72 | | |
| 32 | 2 QD | 286 | 504 | — | 49 | 2 | 4 |
| | | 251 | 288 | 145 | 48 | | |
| | | 252 | 242 | 149 | 54 | | |
| 33 | 3 QD | 226 | 164 | 168 | 25 | 12 | — |
| | | 163 | 82 | 116 | 10 | | |
| 34 | 2 QD | 294 | 194 | 173 | 58 | −1 | −12 |
| | | 297 | 191 | 193 | 65 | | |

QD = daily quantity of capsules
*WO = without nicotinic acid

### EXAMPLE 2

An alternate exemplary embodiment comprises capsules made in a similar manner to that described above wherein each capsule comprises:
about 300 mg red yeast rice;
about 20 mg nicotinic acid;
about 100 mg L-carnitine;
about 0.15 mg folic acid;
about 0.05 mg chromium picolinate; and
about 0.0125 mg selenium.

Exemplary dosages of the composition are from one (1) to four (4) capsules daily. Thus, exemplary daily intakes range from:
about 300 mg to about 1200 mg red yeast rice;
about 20 mg to about 80 mg nicotinic acid;
about 100 mg to about 400 mg L-carnitine;
about 0.15 mg to about 0.6 mg folic acid;
about 0.05 mg to about 0.2 mg chromium picolinate; and
about 0.0125 mg to about 0.05 mg selenium.

The alternate exemplary embodiment provides a lower dose of niacin (nicotinic acid) in part to reduce side effects such as flushing.

Certain exemplary embodiments of the invention have been described herein. However, it will be appreciated that those skilled in the art, upon consideration of this disclosure may make variations and modifications within the spirit and scope of the invention.

1-2. (canceled)

3. A cholesterol treatment composition comprising:
about 300 mg red yeast rice;
about 20 to about 62.5 mg nicotinic acid:
about 100 mg L-carnitine:
about 0.15 mg folic acid:
about 0.05 mg chromium picolinate; and
about 0.0125 mg selenium:
wherein said composition is in a dosage form,
wherein said dosage form is at least one member selected from the group consisting of a tablet and a capsules,
wherein an effective cholesterol treatment amount comprises 1-4 doses per day.

4. The composition of claim 3 wherein said dosage form is a capsule.

5. The composition of claim 3 further comprising an inactive pharmaceutically acceptable carrier.

6. The composition of claim 3 wherein said red yeast rice is produced from the fermentation of at least one member selected from the group consisting of Monascus purpureus, Monascus rubber, Monascus fuliginosus, Monascus Pilosus, and Monascus albidus.

7. The composition of claim 3 comprising:
about 300 mg red yeast rice;
about 20 mg nicotinic acid;
about 100 mg L-carnitine;
about 0.15 mg folic acid;
about 0.05 mg chromium picolinate; and
about 0.0125 mg selenium.

8. The composition of claim 3 comprising:
about 300 mg red yeast rice;
about 62.5 mg nicotinic acid;
about 100 mg L-carnitine;
about 0.15 mg folic acid;
about 0.05 mg chromium picolinate; and
about 0.0125 mg selenium.

9. The composition of claim 7 further comprising an inactive pharmaceutically acceptable carrier, and wherein said dosage form is a capsule.

10-15. (canceled)

16. The composition of claim 9 wherein said capsule comprises:
about 300 mg red yeast rice, about 20 mg nicotinic acid, about 100 mg L-carnitine, about 0.15 mg folic acid, about 0.05 mg chromium picolinate, and about 0.0125 mg selenium.

17. The composition of claim 9 wherein said capsule comprises:
about 300 mg red yeast rice,
about 62.5 mg nicotinic acid,
about 100 mg L-carnitine,
about 0.15 mg folic acid,
about 0.05 mg chromium picolinate, and
about 0.0125 mg selenium.

18. A cholesterol treatment dosage including a composition comprising:
about 300 mg red yeast rice,
about 20 mg nicotinic acid,
about 100 mg L-carnitine,
about 0.15 mg folic acid,
about 0.05 mg chromium picolinate,
about 0.0125 mg selenium, and
an inactive pharmaceutically acceptable carrier;
wherein an effective cholesterol treatment amount comprises one to four doses daily.

19. The dosage of claim 18 wherein the effective cholesterol treatment amount comprises two daily doses.

20. The dosage of claim 18 wherein the effective cholesterol treatment amount comprises three daily doses.

21. The dosage of claim 18 comprising either a capsule form or a tablet form.

22. The dosage of claim 21 comprising a capsule form.

23. The dosage of claim 21 comprising a tablet form.

24. A cholesterol treatment dosage including a composition comprising:
about 300 mg red yeast rice,
about 62.5 mg nicotinic acid,
about 100 mg L-carnitine,
about 0.15 mg folic acid,
about 0.05 mg chromium picolinate,
about 0.0125 mg selenium, and
an inactive pharmaceutically acceptable carrier;
wherein an effective cholesterol treatment amount comprises one to four doses daily.

25. The dosage of claim 24 wherein an effective cholesterol treatment amount comprises one daily dose.

26. The dosage of claim 24 comprising either a capsule form or a tablet form.

27. The dosage of claim 26 comprising a capsule form.

28. The dosage of claim 26 comprising a tablet form.
